(19) United States
(12) Patent Application Publication
(10) Pub. No.: US 2010/0030240 A1
(43) Pub. Date: Feb. 4, 2010

(54) CLOSURE APPARATUS

(76) Inventors: Vladimir Brailovski, Montreal (CA); Yannick Baril, Montreal (CA); Patrick Terriault, Verdun (CA); Raymond Cartier, Montreal (CA)

Correspondence Address: Tessier & Associes;C P 54029, Mount-Royal, Quebec, H1P 3H4, CA

(21) Appl. No.: 12/448,532

(22) PCT Filed: Dec. 24, 2007
(86) PCT No.: PCT/CA2007/002361

§ 371 Date: Jun. 24, 2009

Related U.S. Application Data
(60) Provisional application No. 60/876,993, filed on Dec. 26, 2006.

(51) Int. Cl. A61B 17/04

(52) U.S. Cl. 606/144

(57) ABSTRACT

An apparatus for binding together two biological tissue portions with a binding component. The apparatus includes a body defining a body handle for receiving a hand; at least one drum rotatably mounted to the body, the at least one drum including a binding component attachment for attaching the binding component thereto; a drum actuating lever mounted to the body, the drum actuating lever being operatively coupled to the at least one drum for rotating the at least one drum in a predetermined direction when the drum actuating lever is moved; and a crimping assembly operatively coupled to the body for holding a crimp component and selectively crimping the crimp component to the binding component. The body handle, the drum actuating lever and the crimping assembly are configured, sized and positioned in a manner such that the intended user is able to tighten the binding component around the at least one drum by moving the drum actuating lever using one hand; and the intended user is able to operate the crimping assembly to selectively crimp the crimp component to the binding component middle section using another hand while simultaneously holding the body handle with the first hand.

### FIELD OF THE INVENTION

The present invention relates to the general field of closure apparatuses and is particularly concerned with an apparatus suitable for binding together two biological tissue portions with a binding component.

### BACKGROUND OF THE INVENTION

There is a need in many medical interventions to bind together two biological tissue portions so that they can be attached to each other temporarily until they bind together through biological healing processes. For example, when performing thoracic surgery, there is a need to re-attach together two sternum halves until the fracture healing process binds them together.

Because of the relatively large forces that must be exerted in many instances onto a binding component that attaches to each other the two biological tissue portions, a specialized apparatus is often used to apply the required force. However, many currently used apparatuses require that the surgeon use both hands to apply the proper force to the binding component. Therefore, the surgeon is unable to use the other hand to guide the apparatus or perform other tasks while the prior art apparatuses are used. In addition, it often happens that the binding component is fixed to the sternum or any other two biological tissue portions using a crimp component that must be crimped to the binding component. In many cases, there is no single apparatus that allows to both apply the proper tension onto the binding component and to crimp the crimped component to the binding component.

Accordingly, against this background, there exists a need for an improved closure apparatus.

### SUMMARY OF THE INVENTION

In a first broad aspect, the invention provides an apparatus for binding together two biological tissue portions with a binding component. The binding component has a substantially elongated configuration and defines substantially opposed binding component first and second end sections and a binding component middle section extending therebetween. The binding component is securable around the two biological tissue portions with a crimp component. The apparatus is usable by an intended user having first and second hands. The apparatus includes a body defining a body handle for receiving one of the first and second hands; at least one drum rotatably mounted to the body, the at least one drum including a binding component attachment for attaching the binding component first end section thereto; a drum actuating lever mounted to the body, the drum actuating lever being movable between a drum actuating lever first position and a drum actuating lever second position, the drum actuating lever being operatively coupled to the at least one drum for rotating the at least one drum in a predetermined direction when the drum actuating lever is moved from the drum actuating lever first position to the drum actuating lever second position; and a crimping assembly operatively coupled to the body for holding the crimp component and selectively crimping the crimp component to the binding component middle section. The body handle, the drum actuating lever and the crimping assembly are configured, sized and positioned in a manner such that the intended user is able to tighten the binding component around the at least one drum by moving the drum actuating lever between the drum actuating lever first and second positions using the first hand; and the intended user is able to operate the crimping assembly to selectively crimp the crimp component to the binding component middle section using the second hand.

Advantageously, the intended user is able to tighten the binding component around the at least one drum using a single hand, which frees the other hand to perform other tasks and operations. In addition to allowing the intended user to use the second hand to crimp the crimp component to the binding component at a suitable moment during the operation of the proposed apparatus, the intended user may also use the other hand to guide the position of the crimping assembly so as to suitably position the crimp component relatively to the two biological tissue portions and relatively to the binding component to optimize the position of the crimp component. In addition, the second hand may also be used for any other purposes such as, for example, guiding adjacent pieces of biological tissues away from the binding component when tightening the binding component, removing blood from the site at which the crimp component is positioned and any other suitable operation that helps the intended user in performing the binding of the two biological tissue portions to each other rapidly, ergonomically and safely. Therefore, the other hand is usable to enhance the safety of the procedure performed using the proposed apparatus.

In some embodiments of the invention, a clutch is provided for limiting a torque that the drum may exert onto the binding component. In turn, this ensures that excessive pressure is not exerted onto the two biological tissue portions, which facilitates healing. In some embodiments of the invention, the binding component used with the proposed apparatus includes a shape-memory alloy and the clutch is therefore, in these embodiments, advantageous in allowing the intended user to tighten the binding component with a force that remains below a damage threshold at which the force exerted onto the binding component would permanently damage the binding component.

The proposed apparatus is manufacturable using known components and methods at relatively low costs. Furthermore, the proposed apparatus is manufacturable using materials that are relatively easily sterilizable. Yet, furthermore, the proposed apparatus is usable using a relatively small number of quick and ergonomic steps.

### BRIEF DESCRIPTION OF THE DRAWINGS

An embodiment of the present invention will now be disclosed, by way of example, in reference to the following drawings in which:

FIG. 1, in a side elevation view, illustrates and apparatus for binding together two biological tissue portions (not shown in FIG. 1), the apparatus being usable with a binding component shown in phantom lines;

FIG. 2, in a top elevation view with portions removed, illustrates the apparatus shown in FIG. 1;

FIG. 3, in a side partial cross-sectional view, illustrates the apparatus shown in FIGS. 1 and 2;

FIG. 4, in a front elevation view with portions removed, illustrates the apparatus shown in FIGS. 1 to 3, the apparatus being shown binding together two biological tissue portions using a binding component;

FIG. 5, in a side elevation view, illustrates a drum of the apparatus shown in FIGS. 1 to 4, the drum being shown with a binding component attachment thereof in an attachment closed configuration;

FIG. 6, in a side elevation view, illustrates the drum shown in FIG. 5, the drum being shown with the binding component attachment thereof in an attachment opened configuration;

FIG. 7, in a side elevation view with portions removed, illustrates a crimping assembly of the apparatus shown in FIGS. 1 to 4, the crimping assembly including a pair of jaws, the jaws being shown in an opened configuration;

FIG. 8, in a side elevation view with portions removed, illustrates the crimping assembly shown in FIG. 7, the crimping assembly being shown with the jaws thereof in a closed configuration;

FIG. 9, in a side elevation view, illustrates a binding component and a crimp component usable with the apparatus shown in FIGS. 1 to 8;

FIG. 10, in a side cross-sectional view, illustrates the crimp component shown in FIG. 9;

FIG. 11, in a side cross-sectional view, illustrates an alternative crimp component usable with the apparatus shown in FIGS. 1 to 8 and with the binding component shown in FIG. 9;

FIG. 12, in a schematic view, illustrates successive steps in the attachment of two biological tissue portions using the binding and crimp components shown in FIGS. 9 and 10;

FIG. 13, in a schematic view, illustrates a driving assembly for driving the drums of an apparatus according to an alternative embodiment of the invention;

FIG. 14, in a partial side cross-sectional view with portions removed, illustrates the apparatus shown in FIGS. 1 to 8, the apparatus including two jaws, the two jaws being shown in a jaw opened configuration; and

FIG. 15, in a partial side cross-sectional view with portions removed, illustrates the apparatus shown in FIGS. 1 to 8, the apparatus including two jaws, the two jaws being shown in a jaw closed configuration.

### DETAILED DESCRIPTION

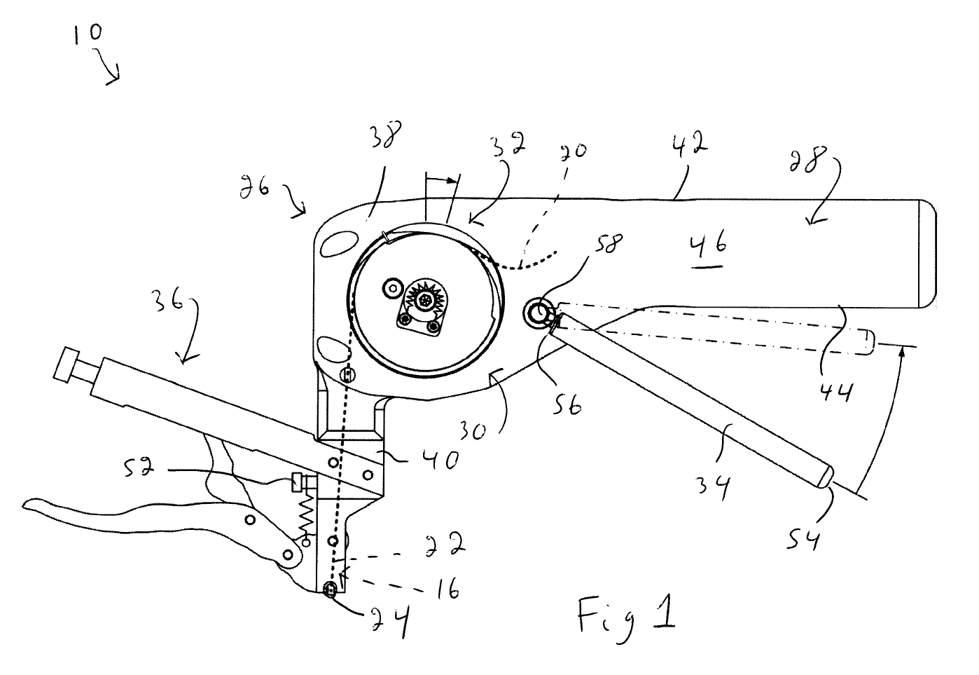

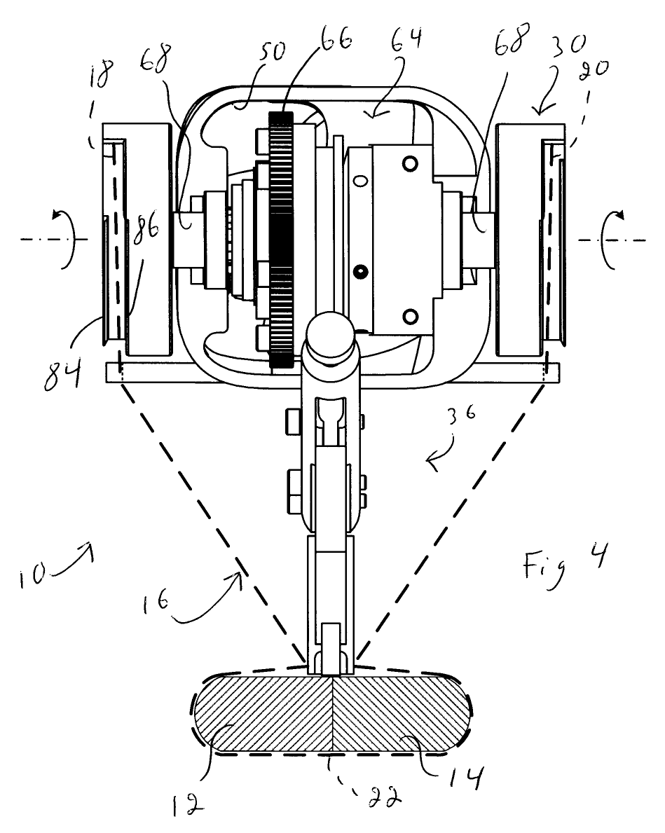

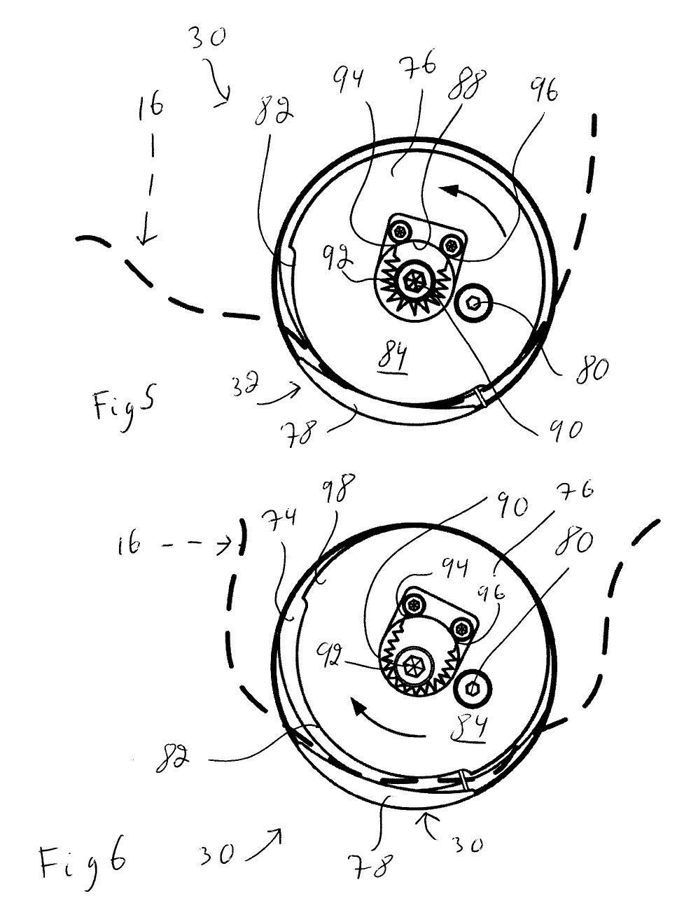

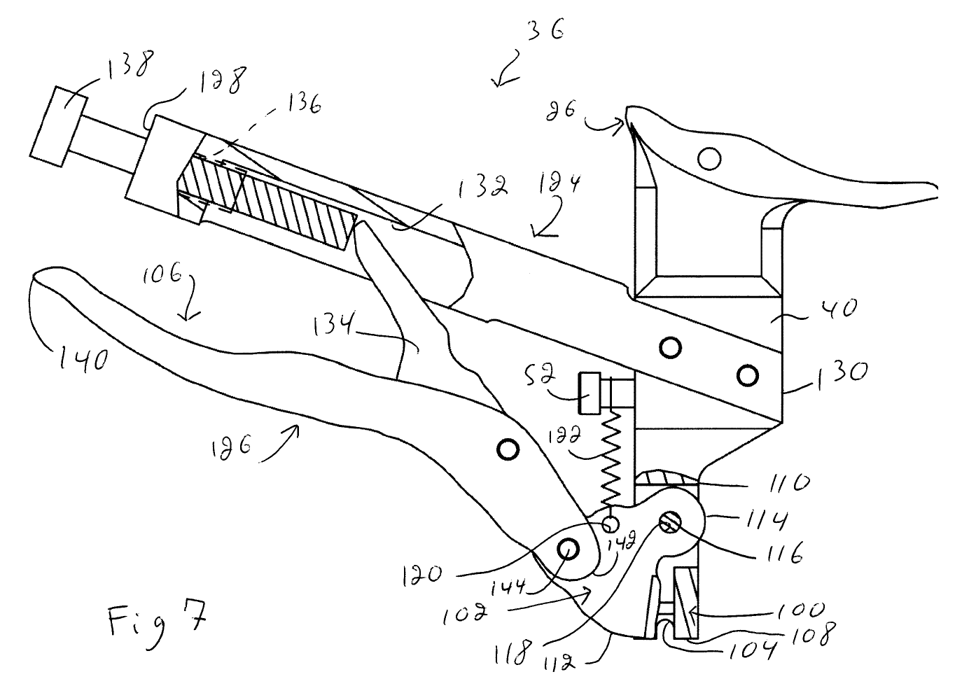

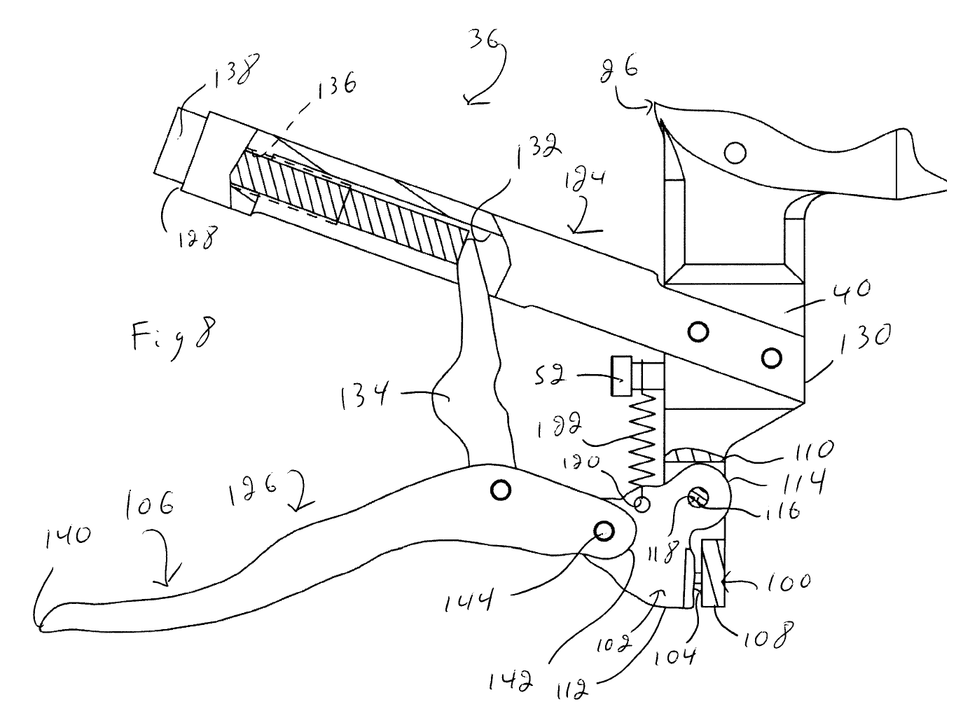

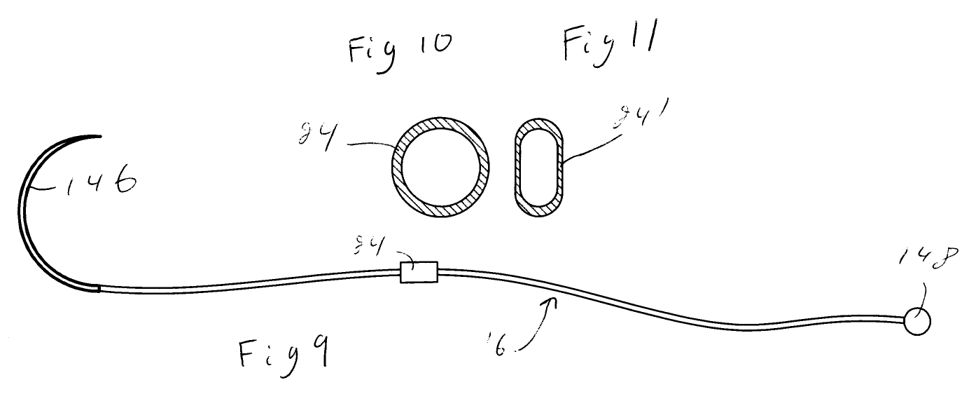

Referring to FIG. 1, there is shown an apparatus 10. Referring to FIG. 4, the apparatus 10 is usable for binding together two biological tissue portions 12 and 14 with a binding component 16 (shown in phantom lines). The binding component 16 has a substantially elongated configuration and defines substantially opposed binding component first and second end sections 18 and 20 and a binding component middle section 22 extending therebetween. As seen in FIG. 12, the binding component 16 is securable around the two biological tissue portions 12 and 14 with a crimp component 24. The apparatus 10 is usable by an intended user having two hands (not shown in the drawings).

Returning to FIG. 1, the apparatus 10 includes a body 26 defining a body handle 28 for receiving the first hand. The apparatus 10 also includes at least one drum 30 and, typically, a pair of substantially opposed drums 30 (only one of which is shown in FIG. 1) rotatably mounted to the body 26. Each of the drums 30 includes a binding component attachment 32 for attaching the binding component 16 thereto. More specifically, each of the binding component attachments 32 is usable for attaching a respective one of the binding component first and second end sections 18 and 20 thereto (only one of which being shown in FIG. 1).

The apparatus 10 also includes a drum actuating lever 34 mounted to the body 26. The drum actuating lever 34 is movable between a drum actuating lever first position, shown in full lines in FIG. 1, and a drum actuating lever second position, shown in phantom lines in FIG. 1. The drum actuating lever 34 is operatively coupled to the drums 30 for rotating the drums 30 in a predetermined direction when the drum actuating lever 34 is moved from the drum actuating lever first position to the drum actuating lever second position.

The apparatus 10 also includes a crimping assembly 36 operatively coupled to the body 26 for holding the crimp component 24 and selectively crimping the crimp component 24 to the binding component 16 in the binding component middle section 22.

The body handle 28, the drum actuating lever 34 and the crimping assembly 36 are configured, sized and positioned in a manner such that the intended user is able to tighten the binding component 16 around the at least one drum 30 by moving the drum actuating lever 34 between the drum actuating lever first and second positions using a first hand and to operate the crimping assembly 36 to selectively crimp the crimp component 24 to the binding component middle section 22 using the second hand.

In addition to the body handle 28, the body 26 defines a body central section 38 from which the body handle 28 extends and a crimping assembly spacing segment 40 extending from the body central section 38. Typically, the crimping assembly spacing segment 40 is angled relatively to the body handle 28. For example, it has been found that having a crimping assembly spacing segment 40 that extends substantially perpendicularly to the body handle 28 provides good ergonomics to the apparatus 10 as it facilitates positioning of the crimp component 24 relatively to the two biological tissue portions 12 and 14 while facilitating handling of the apparatus 10 and crimping of the crimp component 24 to the binding component 16.

The body handle 28 has a substantially elongated configuration and defines a handle first surface 42 and a substantially opposed handle second surface 44. Handle lateral surfaces 46 and 48 (only one of which is shown in FIG. 1) extend between the handle first and second surfaces 42 and 44. The handle second surface 44 is substantially closer to the crimping assembly 36 than the handle first surface 42.

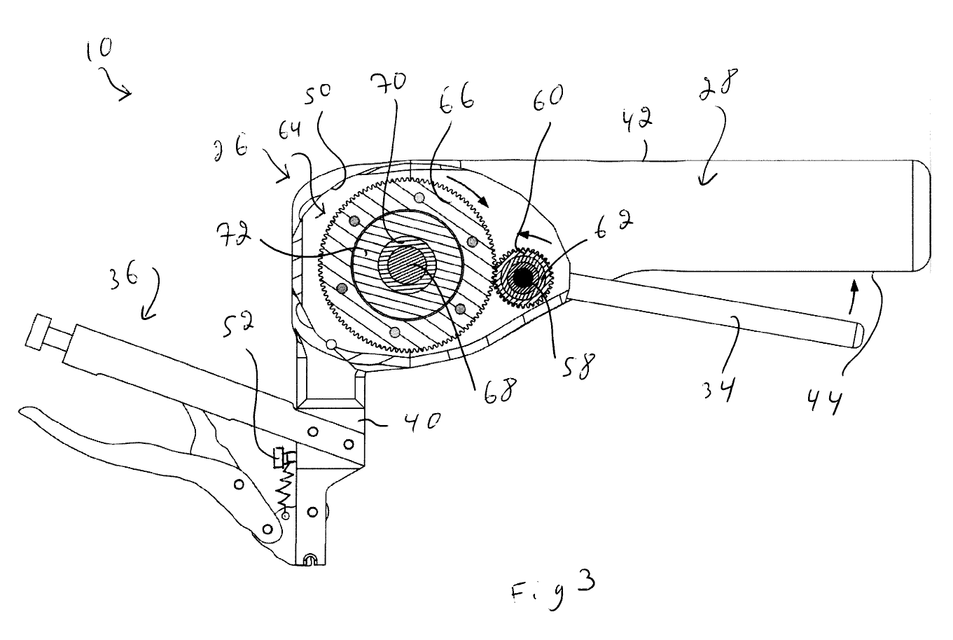

Typically, the body central section 38 defines a central section cavity 50, shown in FIG. 3, for receiving components that couple the drum actuating lever 34 to the drums 30, as described in further details hereinbelow.

The crimping assembly spacing segment 40 is also substantially elongated and typically defines a pin 52 extending substantially adjacent to the crimping assembly 36. The pin 52 is described in further details hereinbelow.

As shown for example in FIG. 1, the drum actuating lever 34 is operatively coupled to the drum 30 so as to be movable from the drum actuating lever second position to the drum actuating lever first position with the drums 30 remaining substantially fixed relatively to the body. Typically, the drum actuating lever 34 is substantially freely movable from the drum actuating lever second position to the drum actuating lever first position with the drums 30 remaining fixed relatively to the body 26. An example of a manner in which this result is achievable is described in further details herienbelow.

The drum actuating lever 34 is pivotally attached to the body 26 substantially adjacent the handle second surface 44. Typically, the drum actuating lever 34 is pivotally attached to the body 26 so as to pivot substantially perpendicularly to the handle second surface 44 when moving between the drum actuating lever first and second positions.

For example, the drum actuating lever 34 defines a drum actuating lever proximal end 54 and a substantially opposed drum actuating lever distal end 56. The drum actuating lever 34 is pivotally attached to the body 26 substantially adjacent the drum actuating lever distal end 56. The drum actuating lever proximal end 54 is typically closer to the handle second surface 44 when the drum actuating lever is in the drum actuating lever second position than when the drum actuating lever 34 is in the drum actuating lever first position. Typically, the drum actuating lever 34 is pivotally attached to the body 26 so as to extend substantially parallel to the body handle 28 when the drum actuating lever is in one of the drum actuating lever first and second positions, and typically in the drum actuating lever second position. This specific configuration enhances the range of motion through which the drum actuating lever 34 may be operated to tighten the binding component 16 using a single hand and facilitates operation and application of a force by the intended user with a single hand so as to optimize force transfer between the hand and the binding component 16.

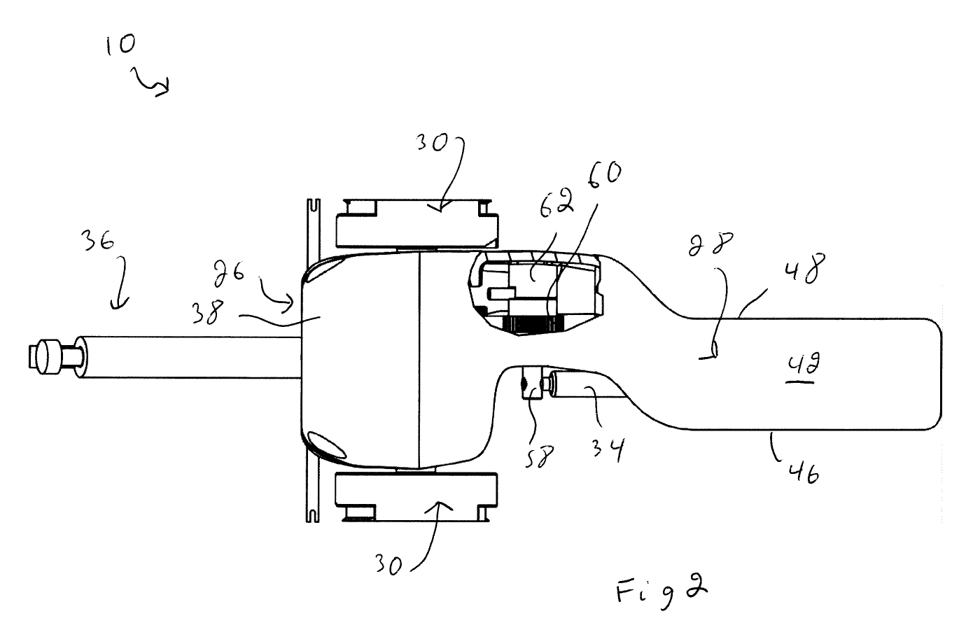

A drum actuating lever mounting axle 58 extends from the drum actuating lever 34 substantially perpendicularly to a direction leading from the drum actuating lever proximal end 54 and to the drum actuating lever distal end 56 and extends substantially adjacent the drum actuating lever distal end 56. The drum actuating lever 34 pivots between the actuating lever first and second positions by rotating about the drum actuating lever mounting axle 58. As seen in FIG. 2, the drum actuating lever mounting axle 58 extends into the central section cavity 50 and is mechanically coupled to a toothed gear 60 for rotating the toothed gear 60 in a predetermined direction. Also, the drum actuating lever mounting axle 58 is mechanically coupled to a roller clutch 62 that allows rotation of the toothed gear 60 in a single direction, for example, when the drum actuating lever 34 is moved from the drum actuating lever first position to the drum actuating lever second position. To that effect, the roller clutch 62 is fixedly mounted to the body 62.

In some embodiments of the invention, the roller clutch 62 allows the drum actuating lever 34 to move substantially freely from the drum actuating lever second position to the drum actuating lever first position. When the apparatus 10 is used with the handle second surface 44 facing generally downwardly, this movement of the drum actuating lever 34 is performed under the action of gravity, which facilitates the operation of the drum actuating lever 34 with a single hand.

Referring to FIG. 3, a power transmission mechanism 64 is mounted inside the central section cavity 50 and operatively coupled to the drums 30 (not shown in FIG. 3) and to the toothed gear 60 for transmitting forces exerted by the intended user onto the drum actuating lever 34 to the drums 30. The power transmission mechanism 64 functions as a torque multiplier to multiply the torque exerted by the intended user onto the drum actuating lever 34 and only allows rotation of the drums 30 in a predetermined direction so that when tension is first applied to the binding component 16, moving the drum actuating lever 34 from the drum actuating lever second position to the drum actuating lever first position does not result in tension inside the binding component 16 to be reduced.

More specifically, this is achieved by having a power transmission mechanism 64 that includes a toothed gear 66 engaging the toothed gear 60 that is coupled to the drum actuating lever 34 for transmitting a rotational motion of the toothed gear 60 to the toothed gear 66. In addition, the power transmission mechanism 64 includes a power transmitting axle 68 for transmitting a rotational motion of the toothed gear 66 to the drums 30. The power transmitting axle 68 is rotatably mounted inside the central section cavity 50 and protrudes laterally therefrom. Each of the drums 30 is mechanically coupled to the power transmitting axle 68 for joint rotation therewith.

A torque limiting clutch 70 and another roller clutch 72 are operatively coupled to the toothed gear 66 and to the power transmitting axle 68 for preventing the power transmitting axle 68 from rotating when a maximal torque has been applied thereto and for only allowing movement of the power transmitting axle 68 in a single direction corresponding to the predetermined direction in which the drums 30 are allowed to rotate. The roller clutches 62 and 72 are configured such that the toothed gears 60 and 66 can rotate in opposite directions.

Roller clutches 62 and 72 and the torque limiting clutch 70 are components that are well known in the art and the specific arrangement used in the apparatus 10 is therefore not described in further details. In addition, in alternative embodiments of the invention, any other suitable components performing similar functions are used instead of the roller clutches 62 and 72 and the torque limiting clutch 70.

Referring to FIGS. 5 and 6, each of the drums 30 includes a drum body 74. The drum body 74 is attached to the power transmitting axle 68 (not shown in FIGS. 5 and 6) for joint rotation therewith. The binding component attachment 32 is mounted to the drum body 74. A specific example of implementation of the binding component attachment 32 is described in further details hereinbelow.

In this specific example, the binding component attachment 32 includes an attachment first member 76 and an attachment second member 78. The attachment first and second members 76 and 78 are operatively coupled to the drum body 74 in a manner such that the binding component attachment 32 is configurable between an attachment opened configuration, shown in FIG. 6, an attachment closed configuration, shown in FIG. 5. In the attachment opened configuration, the attachment first and second members 76 and 78 are substantially spaced apart from each other for allowing inserting of the binding component 16 therebetween. In the attachment closed configuration, the attachment first and second members 76 and 78 are substantially adjacent to each other for receiving the binding component therebetween and frictionally engaging the binding component 16. In some embodiments of the invention, the binding component includes a biasing element operatively coupled to the attachment first and second members 76 and 78 for biasing the attachment first and second members 76 and 78 towards the attachment closed configuration.

In a specific example of implementation, the attachment first member 76 is substantially annular and substantially eccentrically pivotally attached to the drum body 74 through a first member pivot 80. The attachment first member 76 defines a first member peripheral surface 82 and two substantially opposed first member lateral surfaces 84 and 86, only one of which is shown in FIGS. 5 and 6. Also, the attachment first member 76 defines a first member aperture 88 extending between the first member lateral surfaces 84 and 86 in a substantially spaced apart location relatively to the first member pivot 80. Typically, the attachment first member 76 pivots relatively to the drum body 74 about the first member pivot 80 in a plane substantially parallel to a plane in which the drum 30 rotates relatively to the body 26.

The attachment second member 78 is fixed relatively to the drum body 74. Typically, the attachment second member 78 extends integrally from the drum body 74 and is configured and located so that the first member peripheral surface 82 is partially substantially in register therewith.

A pin 90 extends from the drum body 74 through the first member aperture 88. Typically, the pin 90 is co-axial with the power transmitting axle 68. A biasing element in the form of a coil spring 92 biases the attachment first and second members 76 and 78 towards the attachment closed configuration. The coil spring 92 defines a coil spring first end 94 and a coil spring second end 96. The coil spring first and second ends 94 and 96 are secured to the attachment first member 76 with the coil spring 92 wrapping at least partially around the pin 90. The coil spring first and second ends 94 and 96 are located further away from the attachment second member 78 than the pin 90. In this configuration, the coil spring 92 therefore wraps around the pin 90 in a substantially U-shaped configuration. However, in alternative embodiments of the invention the attachment first and second members 76 and 78 are biased towards the attachment closed configuration in any other suitable manner.

In some embodiments of the invention, the attachment first member 76 defines a first member flange 98 extending substantially radially outwardly therefrom substantially adjacent the first member lateral surface 84. The first member flange 98 guides the binding component 16 as it is rolled around the drum 30. The first member flange 98 extends along an arc segment and is located substantially opposed to the attachment second member 78. Typically, the first member flange 98 is absent from locations adjacent the attachment second member 78 to facilitate insertion of the binding component 16 between the attachment first and second members 76 and 78.

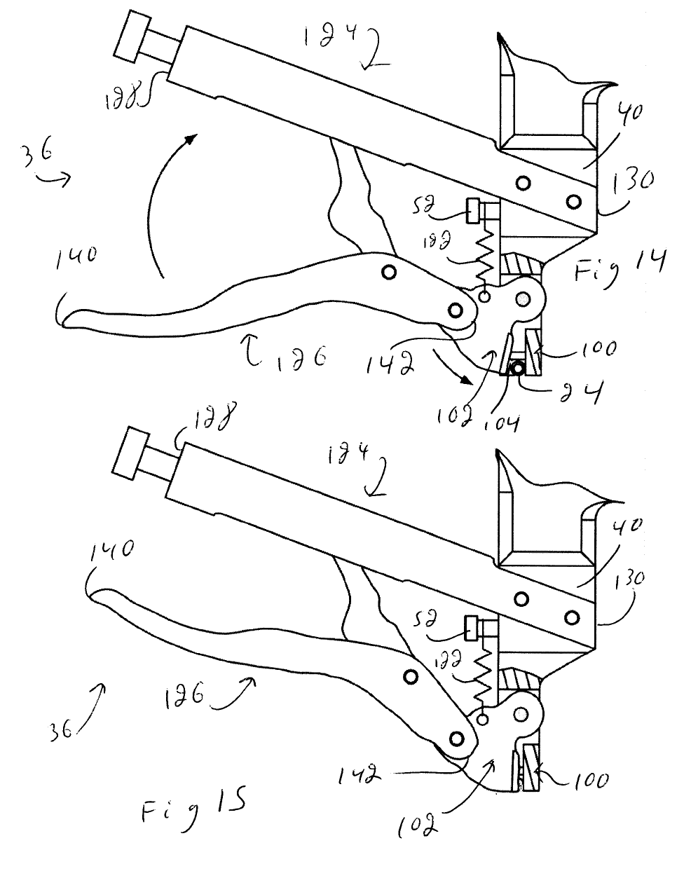

Referring to FIGS. 14 and 15, there is shown in greater details the crimping assembly 36. The crimping assembly 36 includes a pair of jaws 100 and 102. The jaws 100 and 102 are movable between a jaw opened configuration, shown in 14, and a jaw closed configuration, shown in FIG. 15. The jaws 100 and 102 are substantially spaced apart from each other by a larger distance in the jaw opened configuration than in the jaw closed configuration. Referring to FIG. 14, the jaws 100 and 102 define a crimp component holding recess 104 for holding the crimp component 24 when the jaws 100 and 102 are in the jaw opened configuration. The jaws 100 and 102 crimp the crimp component 24 when moved from the jaw opened configuration to the jaw closed configuration. The crimping assembly also includes a crimp actuator 106 operatively coupled to the body 26 and to the jaws 100 and 102 for selectively moving the jaws 100 and 102 between the jaw opened and closed configurations.

Typically, the jaws 100 and 102 include a fixed a jaw, for example the jaw 100, and a mobile jaw, for example the jaw 102. Referring to FIG. 7, the fixed jaw 100 defines a fixed jaw proximal end 108 and a substantially opposed fixed jaw distal end 110. The fixed jaw 100 is fixedly attached to the body 26 and, more specifically, to the crimping assembly spacing segment 40, substantially adjacent the fixed jaw distal end 110.

The mobile jaw 102 defines a mobile jaw proximal end 112 and a substantially opposed mobile jaw distal end 114. The mobile jaw 102 is pivotally attached to the fixed jaw 100 with the fixed jaw and mobile jaw distal ends 110 and 114 substantially adjacent to each other. For example, this is performed by having a fixed jaw 100 that defines a pin 116 extending generally parallel to the power transmitting axle 68 (not seen in FIGS. 7 and 8) and a mobile jaw 102 that defines a pin receiving aperture 118, the pin 116 being inserted through the pin receiving aperture 118.

In some embodiments of the invention, the mobile jaw 102 defines a biasing component attachment aperture 120 at a location intermediate the mobile jaw proximal and distal ends 112 and 114, the biasing component attachment aperture 120 being usable for attaching a biasing component 122 to the mobile jaw 102 as described in further details hereinbelow.

Having a fixed jaw 100 and a mobile jaw 102, as opposed to having two mobile jaws, contributes to the ease of use of the apparatus 10 and facilitates positioning of the crimp component 24 relatively to the two portions of biological tissues 12 and 14 that need to be bound to each other. Also, this configuration facilitates the maintenance of the position of the crimp component 24 when the crimp component 24 is crimped to the binding component 16. However, in alternative embodiments of the invention, both of the jaws 100 and 102 are mobile relatively to the body 26.

A specific configuration of the crimp actuator 106 is described hereinbelow. In this specific configuration, the crimp actuator 106 includes a crimp handle 124 mechanically coupled to the body 26 and a crimp actuating lever 126 located substantially adjacent to the crimp handle 124. The crimp actuating lever 126 is movable between a crimp actuating lever first position and a crimp actuating lever second position. The crimp actuating lever 126 is operatively coupled to the jaws 100 and 102 in a manner such that the jaw 100 and 102 are in the jaw opened configuration when the crimp actuating lever 126 is in the crimp actuating lever first position, shown in FIG. 14, and the jaws 100 and 102 are in the jaw closed configuration when the crimp actuating lever 126 is in the crimp actuating lever second position, shown in FIG. 15. Also, in some embodiments of the invention, the biasing component 122 takes the form of a coil spring extending between the mobile jaw 102 and the pin 52 for biasing the mobile jaw 102 towards the jaw opened configuration. The biasing component 122 is therefore attached at one end thereof to the biasing component attachment aperture 120 and attached at the other end thereof to the pin 52.

When the crimping assembly 36 is in use, the intended user, (not shown in the drawings), exerts an input force onto the crimping assembly 36 to crimp the crimp component 24 to the binding component 16. In some embodiments of the invention, the manner in which the input force is transmitted to the jaws 100 and 102 is adjustable by having the crimp actuator 106 that is configurable between a crimp actuator low leverage configuration, seen in FIG. 7, and a crimp actuator high leverage configuration, seen in FIG. 8.

The crimp actuator 106 is operatively coupled to the pair of jaws 100 and 102 in a manner such that a larger force is exerted by the crimp actuator onto the jaws 100 and 102 when moving the jaws 100 and 102 from the jaw opened configuration to the jaw closed configuration with the input force when the crimp actuator 106 is in the crimp actuator high leverage configuration than when the crimp actuator 106 is in the crimp actuator low leverage configuration.

The functional characteristics of the crimping assembly 36 described hereinabove are achieved, for example, using the following configuration of the crimping assembly 36. The crimp handle 124 defines a crimp handle proximal end 128 and a crimp handle distal end 130. The crimp handle 124 is attached to the crimping assembly spacing segment 40 substantially adjacent the crimp handle distal end 130 in a substantially space apart relationship relatively to the fixed jaw 100.

For example, the crimp handle extends at an angle of from about 20 to about 40 degrees relatively to the body handle 28. It has been found that this configuration enhances the ergonomics of the apparatus 10 as it facilitates operation of the apparatus 10 by the intended user with one hand remaining on the body handle 28 (not shown in FIGS. 7 and 8) and the other hand operating the crimping assembly 36. The crimp handle 124 also defines a link receiving recess 132 extending substantially longitudinally therealong for receiving a crimp actuator link 134 which is described in further details hereinbelow.

The crimp handle 124 further defines a handle aperture 136 extending substantially longitudinally between the link receiving recess 132 and the crimp handle proximal end 128. The handle aperture 136 is threaded and receives a bolt 138 that is threadably inserted thereinto for movement between a bolt first position, seen in FIG. 7, and a bolt second position, seen in FIG. 8. The bolt 138 is used to move the crimp actuator 106 between the crimp actuator low and high leverage configurations.

The crimp actuating lever 126 defines a crimp actuating lever proximal end 140 and a substantially opposed crimp actuating lever distal end 142. The crimp actuating lever 126 is pivotally attached to the mobile jaw 102 with a crimp actuating lever-to-jaw pivot 144. The crimp actuating lever-to-jaw pivot 144 is located substantially adjacent to the crimp actuating lever distal end 142 and to the mobile jaw proximal end 112.

The crimp actuator link 134 extends between the crimp actuating lever 126 and the crimp handle 124. The crimp actuator link 134 is pivotally mounted to both the crimp handle 124 and the crimp actuating lever 126. The crimp actuator link 134 is pivotally mounted to the crimp actuating lever 126 at a location intermediate the crimp actuating lever distal and proximal ends 142 and 140. The crimp actuator link 134 is received inside the link receiving recess 132 and abuts against and is biased towards the bolt 138. Therefore, the crimp actuator link 134 is mounted to the crimp handle 124 so that an end section thereof is selectively movable substantially longitudinally therealong. This movement allows an achievement of the crimp actuator high and low leverage configurations when the bolt 138 is moved in the handle aperture 136.

Referring to FIG. 9, there is shown a typical configuration of the binding component 16 and the crimp component 24 that are usable with the apparatus 10. As seen in FIG. 9, typically, a needle 146 is attached to the binding component 16 substantially adjacent one of the ends thereof. The needle 146 facilitates the insertion of the binding component 16 through the crimp component 24 and around the two biological tissue portions 12 and 14. In some embodiments of the invention, a stopper 148 is attached to the binding component 16 substantially adjacent the other end thereof.

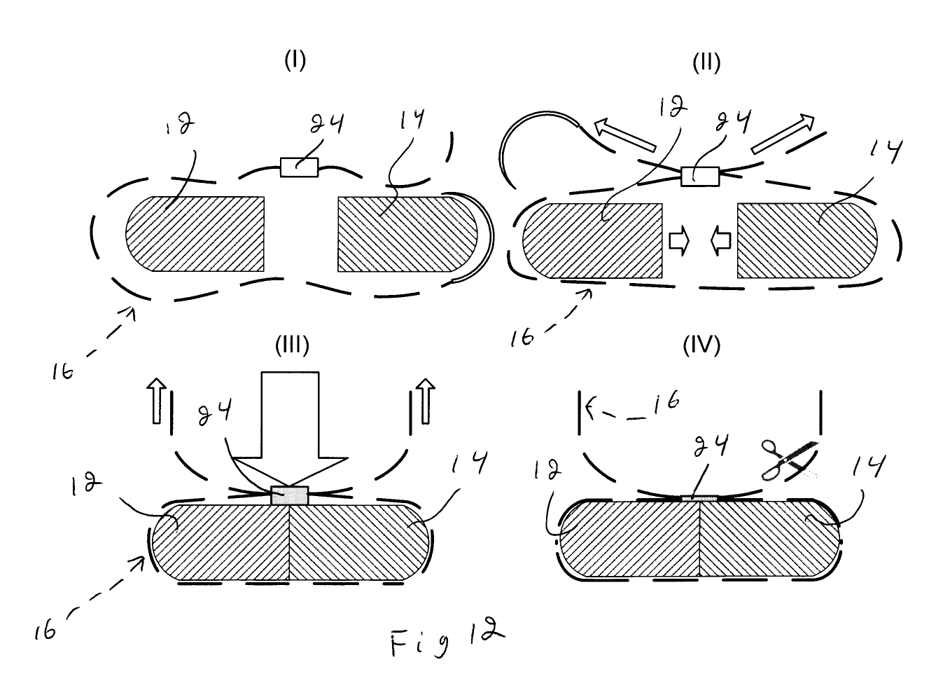

As seen in FIG. 10, in some embodiments of the invention, the crimp component 24 has a substantially annular cross-sectional configuration. However, in alternative embodiments of the invention, a crimp component 24′ shown in FIG. 11 having a substantially oblong tubular configuration is usable. This alternative crimp component 24′ is, in some embodiments, more easily positionable between the jaws 100 and 102 and more easily holdable therebetween before crimping.

FIG. 12 illustrates schematically a manner in which the binding component 16 is usable to bind the two biological tissue portions 12 and 14 thereto. First, as seen in panel (I), the binding component 16 is inserted through the crimp component 24 and passed around the two biological tissue portions 12 and 14 to bind. Afterwards, as seen in panel (II), the binding component 16 is passed back through the crimp component 24 in the same direction in which it was originally passed therethrough to form a loop and a force is exerted on the two end portions of the binding component 16 to pull the two biological tissue portions 12 and 14 towards each other. As seen in panel (III), when a desired tension has been achieved in the binding component 16, for example, a predetermined tension, a force is exerted onto the crimp component 24 to crimp the crimp component 24 to the binding component 16 and the binding component 16 is cut substantially adjacent the crimp component 24 to remove its free ends.

The method schematically illustrated in FIG. 12 and generally described hereinabove is facilitated by the use of the apparatus 10. In such a use, after the binding component 16 has been passed twice through the crimp component 24, the crimp component 24 is inserted between the two jaw 100 and 102 and the binding component 16 is attached to each of the drums 30 using the binding component attachments 32. This is achieved by opening a gap between the attachment first and second members 76 and 78 by moving the binding component attachment 32 from the attachment closed to the opened configurations and, afterwards, releasing the binding component attachment 32 so that the attachment closed configuration is achieved through the action of the coil spring 92.

Afterwards, the intended user uses a hand to repetitively move the drum actuating lever 34 from the drum actuating lever first position to the drum actuating lever second position and, in between each of these moves, release the drum actuating lever 34 which, under the action of gravity, moves back to the drum actuating lever first position. This has for effect to repetitively move the drums 30 in the predetermined direction until a maximal torque is achieved.

Then, the intended user may use the crimping assembly 36 to crimp the crimp component 24 to the binding component 16 by moving the crimp actuating lever 126 towards the crimp actuating lever second position, thereby exerting a force pushing the mobile jaw 102 towards the fixed jaw 100. If desired, before this operation is performed, the bolt 138 may be moved in and out of the link receiving recess 132 so that a desired leverage effect is achieved from the crimp actuator link 134 to adjust the force with which the crimp component 24 is crimped to the binding component 16. Afterwards, the crimp actuating lever 126 is released, which opens the jaws 100 and 102 and allows removal of the apparatus 10 from the site of operation after the free ends of the binding component 16 have been cut off.

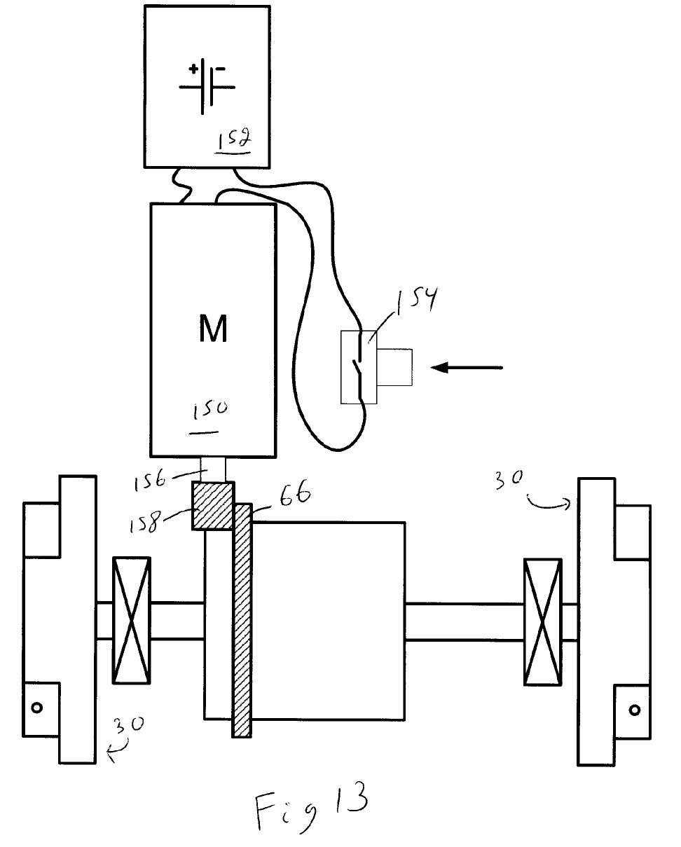

As seen in FIG. 13, in alternative embodiments of the invention, the toothed gear 66, and therefore the drums 30, are rotated by an electric motor 150 connected to a power source 152, for example a battery. To that effect, an output axle 156 is coupled to the electric motor 150 for being rotated thereby. A toothed gear 158 is attached to the output axle 156 for joint rotation therewith. The toothed gear 158 engages the toothed gear 66 and rotates the toothed gear 66 when rotated. A switch 154 allows to selectively energize the electric motor 150 to rotate the toothed gear 66.

Although the present invention has been described hereinabove by way of preferred embodiments thereof, it can be modified without departing from the spirit, scope and nature of the subject invention, as defined in the appended claims.

We claim:

1. An apparatus for binding together two biological tissue portions with a binding component, said binding component having a substantially elongated configuration and defining substantially opposed binding component first and second end sections and a binding component middle section extending therebetween, said binding component being securable around said two biological tissue portions with a crimp component, said apparatus being usable by an intended user having first and second hands, said apparatus comprising:
a body defining a body handle for receiving said first hand;
at least one drum rotatably mounted to said body, said at least one drum including a binding component attachment for attaching said binding component first end section thereto;
a drum actuating lever mounted to said body, said drum actuating lever being movable between a drum actuating lever first position and a drum actuating lever second position, said drum actuating lever being operatively coupled to said at least one drum for rotating said at least one drum in a predetermined direction when said drum actuating lever is moved from said drum actuating lever first position to said drum actuating lever second position; and
a crimping assembly operatively coupled to said body for holding said crimp component and selectively crimping said crimp component to said binding component middle section;
wherein said body handle, said drum actuating lever and said crimping assembly are configured, sized and positioned in a manner such that
said intended user is able to tighten said binding component around said at least one drum by moving said drum actuating lever between said drum actuating lever first and second positions using said first hand; and
said intended user is able to operate said crimping assembly to selectively crimp said crimp component to said binding component middle section using said second hand while simultaneously, holding said body handle with said first hand.

2. An apparatus as defined in claim 1, wherein said drum actuating lever is operatively coupled to said at least one drum so as to be movable from said drum actuating lever second position to said drum actuating lever first position with said at least one drum remaining substantially fixed relatively to said body.

3. An apparatus as defined in claim 2, wherein said drum actuating lever is substantially freely movable from said drum actuating lever second position to said drum actuating lever first position with said at least one drum remaining fixed relatively to said body.

4. An apparatus as defined in claim 1, wherein said drum actuating lever is pivotally attached to said body so as to extend substantially parallel to said body handle when said drum actuating lever is in one of said drum actuating lever first and second positions.

5. An apparatus as defined in claim 4, wherein said drum actuating lever is pivotally attached to said body so as to extend substantially parallel to said body handle when said drum actuating lever is in said drum actuating lever second position.

6. An apparatus as defined in claim 1, wherein said body handle defines a handle first surface and a substantially opposed handle second surface, said handle second surface being closer to said crimping assembly than said handle first surface, said drum actuating lever being pivotally attached to said body substantially adjacent said handle second surface.

7. An apparatus as defined in claim 6, wherein said drum actuating lever is pivotally attached to said body so as to pivot substantially perpendicularly to said handle second surface when moving between said drum actuating lever first and second positions.

8. An apparatus as defined in claim 7, wherein said drum actuating lever defines a drum actuating lever proximal end and a substantially opposed drum actuating lever distal end, said drum actuating lever being pivotally attached to said body substantially adjacent said drum actuating lever distal end, said drum actuating lever proximal end being closer to said handle second surface when said drum actuating lever is in said drum actuating lever second position than when said drum actuating lever is in said drum actuating lever first position.

9. An apparatus as defined in claim 1, further comprising a clutch operatively coupled to said at least one drum and to said drum actuating lever for preventing said drum from rotating in said predetermined direction when a torque substantially larger than a maximal torque is exerted on said at least one drum.

10. An apparatus as defined in claim 9, wherein said clutch is a variable torque clutch allowing a selective adjustment of said maximal torque.

11. An apparatus as defined in claim 1, said apparatus comprising a pair of substantially opposed drums each rotatably mounted to said body, each of said drums including a binding component attachment for attaching one of said binding component first and second end sections thereto, each of said drums being operatively coupled to said drum actuating lever in a manner such that both of said drums rotate in said predetermined direction when said drum actuating lever is moved from said drum actuating lever first position to said drum actuating lever second position.

12. An apparatus as defined in claim 1, wherein said crimping assembly includes
a pair of jaws, said pair of jaws being movable between a jaw opened configuration and a jaw closed configuration, wherein said jaws are substantially spaced apart from each other by a larger distance in said jaw opened configuration than in said jaw closed configuration, said jaws defining a crimp component holding recess for holding said crimp component when said jaws are in said jaw opened configuration, said jaws crimping said crimp component when moved from said jaw opened configuration to said jaw closed configuration; and
a crimp actuator operatively coupled to said body and to said pair of jaws for selectively moving said pair of jaws between said jaw opened and closed configurations.

13. An apparatus as defined in claim 12, wherein said pair of jaws includes a fixed jaw fixedly attached to said body and a mobile jaw operatively coupled to said fixed jaw in a manner such that said pair of jaws are movable between said jaw opened and closed configurations.

14. An apparatus as defined in claim 12, wherein said crimp actuator includes a crimp handle mechanically coupled to said body and a crimp actuating lever located substantially adjacent to said crimp handle, said crimp actuating lever being moveable between a crimp actuating lever first position and a crimp actuating lever second position, said crimp actuating lever being operatively coupled to said pair of jaws in a manner such that said pair of jaws are in said jaw opened configuration when said crimp actuating lever is in said crimp actuating lever first position and said pair of jaws are in said jaw closed configuration when said crimp actuating lever is in said crimp actuating lever second position.

15. An apparatus as defined in claim 12, wherein said intended user exerts an input force onto said crimping assembly to crimp said crimp component to said binging component, said crimp actuator being configurable between a crimp actuator low leverage configuration and a crimp actuator high leverage configuration, said crimp actuator being operatively coupled to said pair of jaws in a manner such that a larger force is exerted by said crimp actuator onto said pair of jaws when moving said pair of jaws from said jaw opened configuration to said jaw closed configuration with said input force when said crimp actuator is in said high leverage configuration than when said crimp actuator is in said low leverage configuration.

16. An apparatus as defined in claim 12, wherein
said pair of jaws includes a fixed jaw and a mobile jaw, said fixed jaw defining a fixed jaw proximal end and a substantially opposed fixed jaw distal end, said fixed jaw being fixedly attached to said body substantially adjacent said fixed jaw distal end, said mobile jaw defining a mobile jaw proximal end and a substantially opposed mobile jaw distal end, said mobile jaw being pivotally attached to said fixed jaw with said fixed jaw and mobile jaw distal ends substantially adjacent to each other;
said crimp actuating lever defines a crimp actuating lever proximal end and a substantially opposed crimp actuating lever distal end, said crimp actuating lever being pivotally attached to said mobile jaw with a crimp actuating lever-to-jaw pivot, said crimp actuating lever-to-jaw pivot being located substantially adjacent to both said crimp actuating lever distal end and said mobile jaw distal end;
said crimp actuator includes a crimp actuator link extending between said crimp actuating lever and said crimp handle, said crimp actuator link being pivotally mounted to both said crimp handle and said crimp actuating lever, said crimp actuator link being pivotally mounted to said crimp actuating lever at a location intermediate said crimp actuating lever distal and proximal ends.

17. An apparatus as defined in claim 16, wherein said crimp actuator link defines a link end section mounted to said crimp handle so as to be selectively movable substantially longitudinally therealong.

18. An apparatus as defined in claim 12, wherein said crimping assembly includes a biasing component 122 operatively coupled to said pair of jaws for biasing said pair of jaws towards said jaw opened configuration.

19. An apparatus as defined in claim 1, wherein said body defines a crimping assembly spacing segment extending between said at least one drum and said crimping assembly for spacing said crimping assembly and said at least one drum relatively to each other, said crimping assembly spacing segment being angled relatively to said body handle.

20. An apparatus as defined in claim 19, wherein said crimping assembly spacing segment is substantially perpendicular to said body handle.

21. An apparatus as defined in claim 1, wherein
said at least one drum includes a drum body;
said binding component attachment includes an attachment first member and an attachment second member, said attachment first and second members being operatively coupled to said drum body in a manner such that said binding component attachment is configurable between an attachment opened configuration and an attachment closed configuration, wherein in said attachment opened configuration, said attachment first and second members are substantially spaced apart from each other for allowing an insertion of said binding component therebetween and in said attachment closed configuration, said attachment first and second members are substantially adjacent to each other for receiving said binding component therebetween and frictionally engaging said binding component.

22. An apparatus as defined in claim 21, further comprising a biasing element operatively coupled to said attachment first and second members for biasing said attachment first and second members towards said attachment closed configuration.

23. An apparatus as defined in claim 22, wherein
said attachment first member is substantially annular and substantially eccentrically pivotally attached to said drum body at a first member pivot location, said attachment first member defining a first member peripheral surface; and
said attachment second member is substantially fixed relatively to said drum body, said first member peripheral surface being partially substantially in register with said first member peripheral surface.

24. An apparatus as defined in claim 23, wherein
said attachment first member defines substantially opposed first member lateral surfaces and a first member aperture extending therebetween substantially spaced apart from said first member pivot location;
said drum includes a pin extending through said first member aperture;
said biasing element includes a coil spring defining a coil spring first end and a coil spring second end, said coil spring first and second ends being secured to said attachment first member with said coil spring wrapping at least partially around said pin, said coil spring first and second ends being located further away from said attachment second member than said pin.

25. An apparatus as defined in claim 24, wherein said attachment first member defines a first member flange extending substantially radially outwardly therefrom substantially spaced apart from said drum body.
